(19) United States
(12) Patent Application Publication
(10) Pub. No.: US 2026/0345931 A1
(43) Pub. Date: Nov. 5, 2026

(54) INLINE AIRFLOW MONITORING AND FEEDBACK APPARATUS FOR ASSISTED VENTILATION

(71) Applicant: University of Maryland, College Park, College Park, MD (US)

(72) Inventors: Jason ROSE, Severna Park, MD (US); Kelly YEUNG, Severn, MD (US); Kevin AROOM, Silver Spring, MD (US); Martha Wang, Fulton, MD (US)

(21) Appl. No.: 19/665,170

(22) Filed: May 1, 2026

Related U.S. Application Data
(60) Provisional application No. 63/798,222, filed on May 1, 2025.

(51) Int. Cl. A61M 16/00

(52) U.S. Cl. CPC A61M 16/0003; A61M 2016/003

(57) ABSTRACT

In accordance with the present disclosure, a method of assisting ventilation using an inline airflow apparatus includes receiving airflow through a flow channel disposed between a self-inflating bag and a patient interface, measuring a pressure differential across the flow channel using a flow sensing module, determining one or more ventilation parameters including at least one of a flow rate, a delivered volume, and a ventilation rate based on the pressure differential, comparing the ventilation parameters to predefined target parameters, generating one or more feedback signals based on the comparison, and outputting at least one of visual feedback and auditory feedback to guide a user in operating the self-inflating bag.

### CROSS-REFERENCE TO RELATED APPLICATION AND CLAIM OF PRIORITY

This application claims the benefit of, and priority to, U.S. Provisional Patent Application No. 63/798,222 filed on May. 1, 2025, the entire contents of which are hereby incorporated herein by reference.

### TECHNICAL FIELD

The present disclosure relates generally to the field of respiratory support devices and emergency medical ventilation. More specifically, the present disclosure provides systems, devices, and methods for assisted manual ventilation using a bag valve mask, including flow sensing, signal processing, and real-time user feedback to guide ventilation parameters such as breath volume, rate, and mask seal during resuscitative care.

### BACKGROUND

Manual ventilation using a bag valve mask (BVM) is a widely employed technique for providing respiratory support in emergency and pre-hospital settings. BVMs are commonly used by trained medical personnel, such as emergency medical services (EMS), to deliver positive pressure ventilation to patients experiencing respiratory failure. However, improper use of BVMs presents significant clinical risks. In particular, excessive ventilation volume or rapid breathing frequency can lead to pulmonary barotrauma and lung injury due to overinflation. Additional complications include gastric insufflation, which may result in regurgitation and airway obstruction. Safe and effective use of BVMs therefore requires adherence to appropriate ventilation parameters, including controlled breath volume, rate, and proper mask seal, which can be difficult to achieve without adequate training.

The need for effective respiratory support is particularly acute in the context of the ongoing opioid overdose epidemic, where respiratory depression or failure is the primary cause of death. Tens of thousands of overdose-related deaths occur annually in the United States, many of which present opportunities for timely intervention prior to the arrival of trained medical personnel. Although pharmacological intervention with naloxone (Narcan) is widely used, its effectiveness may be limited by factors such as dosage, timing of administration, or the presence of additional substances that also suppress respiration. Consequently, direct ventilatory support remains a critical and often necessary intervention. Emergency medical services (EMS) personnel are trained to provide such support using bag valve masks (BVMs), however, response times can range from several minutes to over ten minutes, creating a window in which bystander intervention may be decisive for patient survival.

Despite this opportunity for early intervention, conventional BVM devices are not well suited for use by untrained individuals. Effective ventilation requires adherence to proper techniques, including controlled compression depth, appropriate breathing cadence, and proper mask positioning to ensure an adequate seal. In high-stress emergency scenarios, bystanders often lack the training or confidence to perform these actions correctly and may freeze or deliver suboptimal ventilation. Improper use can reduce the effectiveness of resuscitation efforts or exacerbate patient harm, including pulmonary barotrauma from overinflation, excessive ventilation rates, or delivery of air into the stomach leading to regurgitation and airway obstruction. Existing BVM systems generally lack integrated guidance or feedback mechanisms capable of assisting users in achieving appropriate ventilation parameters in real time.

Accordingly, there remains a need for accessible, easy-to-use systems that can guide untrained or minimally trained individuals in delivering safe and effective manual ventilation. Such systems would ideally provide real-time feedback on ventilation performance, including breath volume and rate, to improve the quality of bystander intervention during the critical period before professional medical care is available.

### SUMMARY

In accordance with the present disclosure, a method of assisting ventilation using an inline airflow apparatus includes receiving airflow through a flow channel disposed between a self-inflating bag and a patient interface, measuring a pressure differential across the flow channel using a flow sensing module, determining one or more ventilation parameters including at least one of a flow rate, a delivered volume, and a ventilation rate based on the pressure differential, comparing the ventilation parameters to predefined target parameters, generating one or more feedback signals based on the comparison, and outputting at least one of visual feedback and auditory feedback to guide a user in operating the self-inflating bag.

In an aspect, the method may include determining delivered volume by calculating volume based on the measured pressure differential.

In an aspect, the visual feedback may include illuminating a plurality of LEDs in a progressive manner corresponding to increasing delivered volume.

In an aspect, each LED may correspond to a predefined volume increment.

In an aspect, the visual feedback may include indicating a target volume range using at least one visually distinct LED.

In an aspect, the visual feedback may include generating a periodic illumination pattern corresponding to a target ventilation cadence.

In an aspect, the periodic illumination pattern may include an illumination phase, a dimming phase, and a pause phase corresponding to a predefined breathing interval.

In an aspect, the auditory feedback may include generating one or more instructional prompts.

In an aspect, the instructional prompts may include guidance to adjust compression force, ventilation rate, or timing between compressions.

In an aspect, the method may include detecting a deviation from a target ventilation parameter and generating corrective feedback corresponding to the deviation.

In an aspect, the corrective feedback may include indicating insufficient volume delivery, excessive volume delivery, a ventilation rate above a threshold, a ventilation rate below a threshold, an insufficient seal based on a measured pressure or pressure differential below an expected threshold, or an airway obstruction based on a measured pressure or pressure differential above an expected threshold.

In an aspect, the method may include detecting repeated deviations of a same type and generating an escalated feedback instruction.

In an aspect, the method may include generating a confirmation signal upon achieving a target ventilation parameter.

In accordance with the present disclosure, an inline airflow apparatus for assisting ventilation includes a housing defining a flow channel configured to be fluidly coupled between a self-inflating bag and a patient interface, a flow sensing module having a plurality of sensors positioned along the flow channel and configured to measure pressure at one or more locations, a feedback assembly having at least one visual output device and at least one auditory output device, at least one processor, and at least one memory storing instructions that, when executed by the processor, cause the apparatus to receive airflow through the flow channel, measure a pressure differential across the flow channel, determine one or more ventilation parameters including at least one of a flow rate, a delivered volume, and a ventilation rate based on the pressure differential, compare the ventilation parameters to predefined target parameters, generate feedback signals based on the comparison, and output at least one of visual feedback and auditory feedback to guide a user in operating the self-inflating bag.

In an aspect, the flow channel may include a geometry having an inlet section, a converging section, and a diverging section configured to generate the pressure differential.

In an aspect, the plurality of sensors may be positioned to measure pressure at at least two of the inlet section, the converging section, and the diverging section.

In an aspect, the predefined target parameters may include a target ventilation rate and a target delivered volume.

In an aspect, the feedback assembly may include an LED array configured to provide visual feedback indicative of delivered volume.

In an aspect, the LED array may be configured to illuminate progressively in response to increasing delivered volume and to indicate when a target volume has been reached.

In an aspect, the instructions may cause the apparatus to determine a ventilation rate based on temporal variations in the measured pressure differential and to generate feedback signals corresponding to the ventilation rate.

### BRIEF DESCRIPTION OF THE DRAWINGS

A better understanding of the features and advantages of the present disclosure will be obtained by reference to the following detailed description that sets forth illustrative embodiments, in which the principles of the present disclosure are utilized, and the accompanying drawings of which:

FIG. 1 illustrates a diagram of an inline airflow apparatus and self-inflating bag, in accordance with the present disclosure;

FIG. 2 illustrates a block diagram of a controller of the inline airflow apparatus of FIG. 1, in accordance with the present disclosure;

FIG. 3 illustrates an exploded perspective view of the inline airflow apparatus of FIG. 1, in accordance with the present disclosure;

FIG. 4 illustrates a cross-sectional view of a flow channel of the inline airflow apparatus of FIG. 1, in accordance with the present disclosure;

FIG. 5 illustrates an example visual feedback interface of the inline airflow apparatus of FIGS. 1, 3, and 4, in accordance with the present disclosure;

FIG. 6 illustrates a perspective view of an inline airflow apparatus in use with a self-inflating bag to form a ventilation assembly, in accordance with embodiments of the present disclosure;

FIG. 7 illustrates a cross-sectional perspective view of the inline airflow apparatus of FIG. 6 in use with the self-inflating bag, in accordance with aspects of the present disclosure;

FIG. 8 is a flowchart of an exemplary method for assisting ventilation using the inline airflow apparatus, in accordance with the present disclosure; and

FIGS. 9A-9D are exemplary graphical illustrations of results of comparing ventilation performance with and without use of the inline airflow apparatus, of FIG. 1, in accordance with the present disclosure.

### DETAILED DESCRIPTION

The present disclosure provides systems, devices, and methods for assisted manual ventilation, including flow sensing and real-time feedback to guide ventilation parameters such as breath volume, rate, and mask seal during emergency respiratory support.

Although the present disclosure will be described in terms of specific examples, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

Unless expressly defined herein, all terms used in this disclosure, including the claims, are intended to have their ordinary and customary meaning as understood by a person of ordinary skill in the art to which this subject matter pertains. Where a term is expressly defined, that definition shall govern. Singular forms include plural forms unless the context clearly dictates otherwise, and “comprising” is used in a non-limiting, open-ended sense.

The present disclosure is generally directed to systems and methods for assisted ventilation using a bag valve mask (BVM). More specifically, the present disclosure provides an inline airflow apparatus integrated with a feedback system configured to measure airflow characteristics and provide real-time guidance to a user during manual ventilation. The disclosed system utilizes a flow sensing module positioned within a flow channel to measure a pressure differential generated by airflow, and a controller configured to determine ventilation parameters including flow rate, delivered volume, and ventilation rate. These parameters are compared to predefined target ranges, and corresponding visual and auditory feedback is generated to guide the user toward proper ventilation technique.

Manual ventilation using a BVM is a critical intervention in emergency medicine, including respiratory arrest, cardiac arrest, and opioid overdose scenarios. Effective ventilation requires delivery of air within a target breathing rate and volume range. For example, adult resuscitation guidelines generally recommend a ventilation rate on the order of approximately 12 to 18 breaths per minute and a delivered tidal volume on the order of approximately 400 to 550 mL. However, untrained or minimally trained users often lack the ability to consistently achieve these parameters. Improper ventilation may result in insufficient oxygen delivery, excessive ventilation pressure, or overinflation of the lungs, each of which can negatively impact patient outcomes.

Conventional BVM devices rely entirely on user judgment to determine compression rate and force, and do not provide quantitative feedback regarding delivered airflow or volume. As a result, users may unknowingly ventilate at an incorrect rate, deliver insufficient or excessive volume, or fail to allow adequate refill of the bag between compressions. In addition, proper mask sealing technique, such as the E-C seal, is often difficult for inexperienced users to achieve, resulting in air leakage and reduced ventilation efficiency. These limitations are particularly significant in emergency situations involving untrained bystanders, where rapid and correct intervention is critical but difficult to perform reliably.

The present disclosure addresses these technical shortcomings by introducing a ventilation assistance system that integrates airflow measurement with real-time user feedback. The system employs a flow channel having a geometry configured to generate a pressure differential corresponding to airflow, such as a Venturi-type configuration. A flow sensing module measures the pressure differential across selected sections of the flow channel, and a controller processes the measured data to determine airflow rate, delivered volume, and ventilation timing. By utilizing pressure-based sensing within the airflow path, the system provides a non-invasive and compact solution for real-time monitoring of ventilation performance without requiring additional external instrumentation. The controller is configured to compare the determined ventilation parameters to predefined target parameters and generate feedback signals indicative of user performance. The feedback assembly provides both visual and auditory outputs to guide the user during operation. In some aspects, a visual LED array provides a real-time indication of delivered volume, including progressive illumination corresponding to increasing airflow and color-coded indicators representing under-delivery, target achievement, and over-delivery conditions. In addition, the LED array may provide cadence guidance corresponding to a target ventilation rate. The auditory feedback system provides instructional prompts and corrective feedback, such as directing the user to adjust compression force or timing. By engaging multiple sensory modalities, the system improves usability and increases the likelihood that users can perform ventilation within clinically acceptable ranges.

In this manner, the disclosed system provides a technical solution to the technical problem of inconsistent and potentially unsafe manual ventilation. By combining airflow sensing, real-time parameter estimation, and multimodal feedback, the system reduces variability in user performance and promotes adherence to recommended ventilation guidelines. The system mitigates risks associated with under-ventilation, such as inadequate oxygen delivery, and over-ventilation, such as excessive airway pressure and lung overexpansion. Furthermore, by providing guided instruction during use, the system reduces reliance on prior training and enables more effective intervention by untrained or minimally trained users in emergency scenarios. The system is further configured to be compact, portable, and compatible with existing BVM devices, allowing it to be integrated into emergency response kits or distributed for use alongside opioid prescriptions or other at-risk populations. By providing an affordable and easy-to-use ventilation assistance solution, the system enhances accessibility to life-saving respiratory support while maintaining technical functionality for monitoring and guiding ventilation performance. In some aspects, the system may be configured to support adjustable target parameters to accommodate different patient populations, including pediatric patients, and may be further adapted to provide configurable feedback modes or language options.

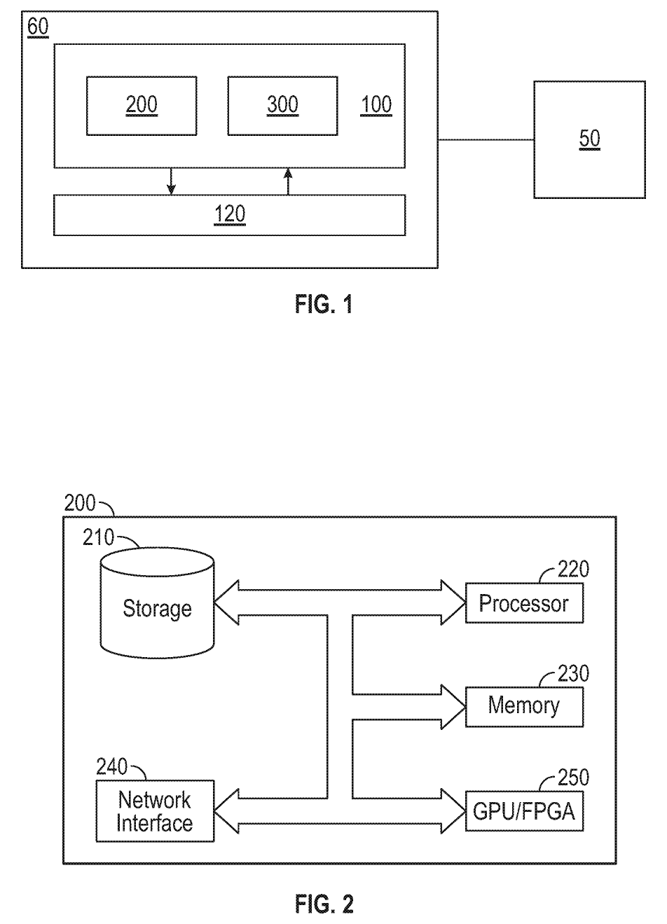

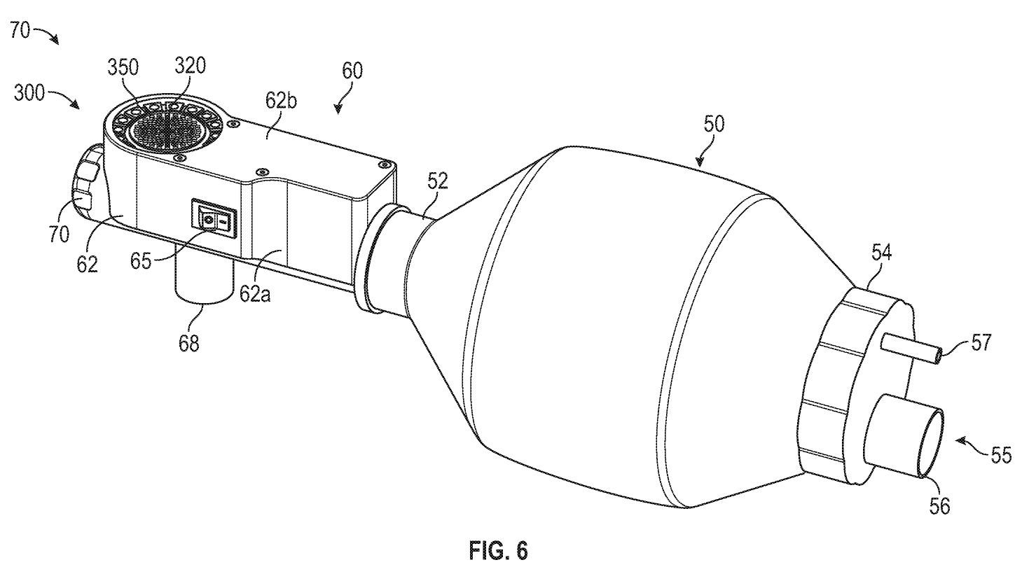

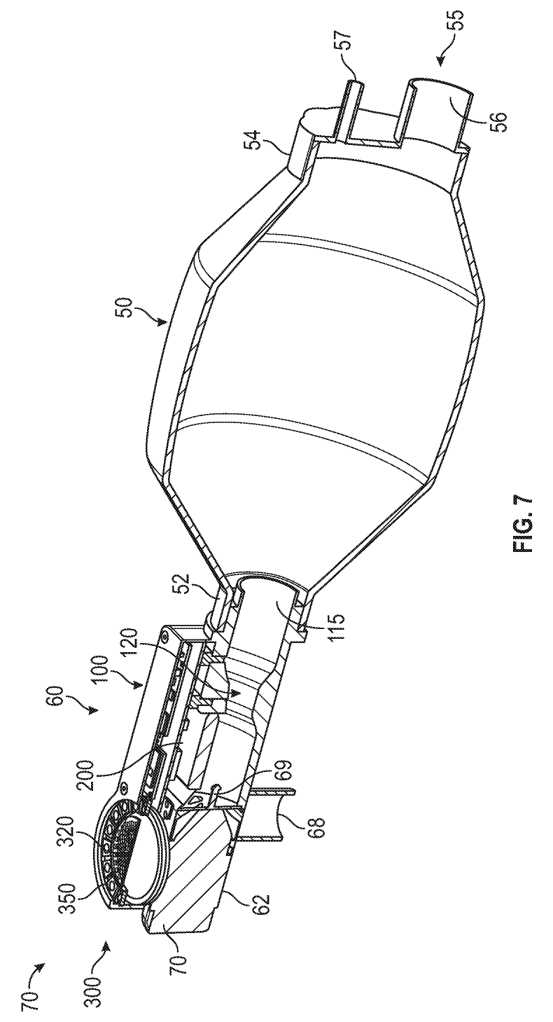

FIG. 1 illustrates an inline airflow apparatus 60 in accordance with the present disclosure. The inline airflow apparatus 60 is configured to measure airflow through a flow channel 120 and to provide real-time feedback to a user based on one or more determined ventilation parameters. In some aspects, the inline airflow apparatus 60 is configured to be fluidly coupled between a ventilation device, such as a self-inflating bag 50, and a patient interface, such that airflow delivered to a patient passes through the inline airflow apparatus 60. An exemplary implementation of the inline airflow apparatus 60 configured for use with a self-inflating bag 50 is illustrated in FIGS. 6-7.

The inline airflow apparatus 60 includes a flow channel 120 configured to define a fluid pathway for airflow between a ventilation device (e.g. self-inflating bag 50) and a patient interface. The inline airflow apparatus 60 further includes a flow sensing module 100, a controller 200, and a feedback assembly 300. The flow sensing module 100 is configured to measure one or more airflow parameters associated with airflow through the flow channel 120, including, for example, flow rate, pressure, and/or tidal volume. In aspects, the flow channel 120 includes a Venturi structure configured to generate a pressure differential corresponding to airflow through the flow channel 120. Sensor data representative of the pressure differential is communicated to the controller 200, which is configured to determine one or more ventilation parameters based on the received signals.

The controller 200 is operably coupled to the feedback assembly 300 and is configured to generate feedback signals based on the determined ventilation parameters. The feedback assembly 300 may include one or more visual indicators, such as light-emitting diodes (LEDs), and/or audio output components configured to provide real-time guidance to a user. In aspects, the feedback assembly 300 provides indications corresponding to ventilation rate and volume, including target thresholds and corrective feedback when measured parameters fall outside desired ranges. In this manner, the inline airflow apparatus 60 is configured to assist a user in delivering ventilation with appropriate cadence and volume during manual operation of the self-inflating bag 50. The inline airflow apparatus 60 is described in more detail below with reference to FIG. 2.

Referring now to FIG. 2, exemplary components of the controller 200 in accordance with aspects of the present disclosure include, for example, a storage module 210, one or more processors 220, at least one memory 230, and a network interface 240. In aspects, the controller 200 may further include a graphical processing unit (GPU) or field-programmable gate array (FPGA) 250, which may be used for processing sensor data or executing computational models.

The processor 220 is configured to execute instructions using data received from the flow sensing module 100, including signals representative of airflow characteristics such as pressure differentials and flow rates. The memory 230 may store transient data, calibration parameters, and operational thresholds, while the storage module 210 may retain historical ventilation data, user settings, and system configurations. The controller 200 processes the received sensor data to determine ventilation parameters, such as breath volume and rate, and compares these parameters to predefined target ranges.

In some aspects, the controller 200 generates feedback signals based on the comparison between measured ventilation parameters and target values. These feedback signals are transmitted to the feedback assembly 300 to provide real-time visual and/or auditory guidance to the user. For example, the controller 200 may cause visual indicators to illuminate in proportion to delivered tidal volume and may generate audio prompts to instruct the user to adjust ventilation rate or compression depth. In this manner, the controller 200 enables dynamic, real-time guidance during manual ventilation.

Database 210 can be located in storage. The term “storage” may refer to any device or material from which information may be capable of being accessed, reproduced, and/or held in a chemical-based digital form, or an electromagnetic or optical form for access by a computer processor. Storage may be, for example, volatile memory such as RAM, non-volatile memory, which permanently holds digital data until purposely erased, such as flash memory, magnetic devices such as hard disk drives, and optical media such as a CD, DVD, Blu-ray disc, or the like. In various aspects, data may be stored on the controller 200, including, for example, user preferences, historical data, and/or other data. The data can be stored in database 210 and sent via the system bus to the processor 220.

As will be described in more detail later herein, the processor 220 executes various processes based on instructions that can be stored in the server memory 230 and utilizing the data from the database 210. The illustration of FIG. 2 is exemplary, and it will be understood by persons skilled in the art that other components may exist in a controller 200. Such other components are not illustrated in FIG. 2 for clarity of illustration.

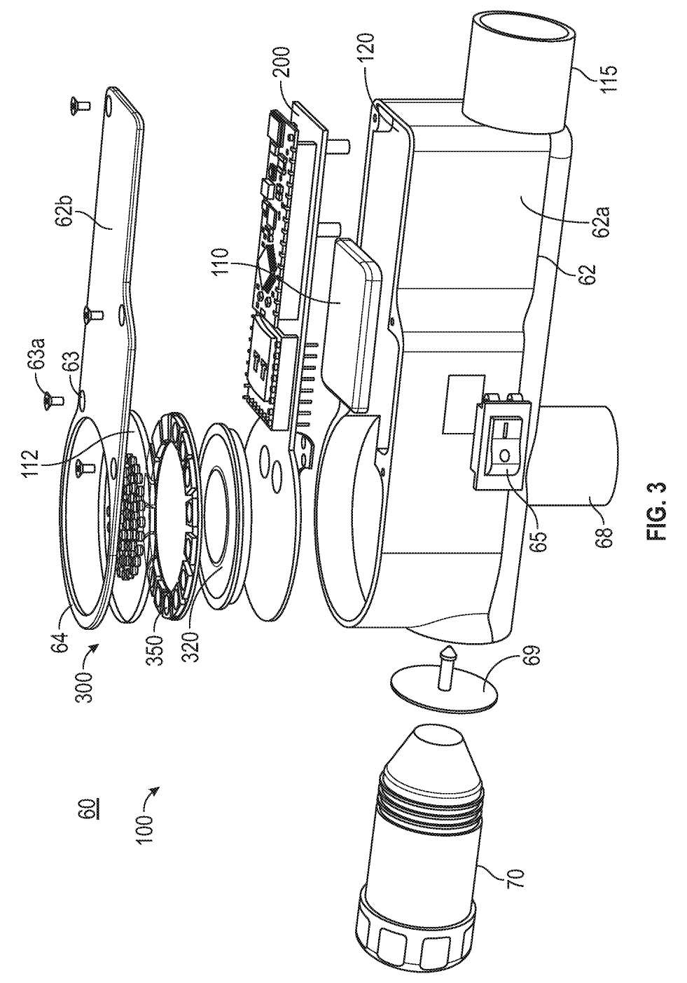

FIG. 3 illustrates an exploded view of the inline airflow apparatus 60 in accordance with aspects of the present disclosure. The inline airflow apparatus 60 includes a housing 62 configured to enclose and support a flow sensing module 100, a controller 200, and a feedback assembly 300. The housing 62 includes a lower housing portion 62a and an upper housing portion 62b that cooperate to define an internal cavity and a flow channel 120 extending between a bag interface port 115 and a patient interface connector 68. The upper housing portion 62b is configured to be secured to the lower housing portion 62a using one or more screw connectors 63a extending through corresponding fastener apertures 63, thereby enclosing the internal components while permitting disassembly for assembly, maintenance, or replacement. The upper housing portion 62b further defines an opening 64 configured to receive the feedback assembly 300.

The lower housing portion 62a defines a primary structural body of the inline airflow apparatus 60 and includes the flow channel 120 formed therein. The flow channel 120 extends longitudinally through the housing 62 and is configured to direct airflow from a ventilation source toward a patient interface. The bag interface port 115 is disposed at a proximal end of the flow channel 120 and is configured to receive a ventilation device, such as a self-inflating bag 50, in a sealed manner (FIGS. 6 and 7). The patient interface connector 68 is disposed at a distal end of the flow channel 120 and is configured to couple to a face mask or airway interface. In aspects, an umbrella valve 69 is positioned along the flow path proximate to the patient interface connector 68 and is configured to permit one-way airflow toward the patient while inhibiting reverse flow.

The housing 62 further defines one or more internal mounting features configured to support components of the inline airflow apparatus 60. A power source 110, such as a battery, is positioned within the housing 62 and is configured to provide electrical power to the controller 200 and feedback assembly 300. A power switch 65 is disposed on an exterior surface of the housing 62 and is electrically coupled to the power source 110 to selectively activate or deactivate the inline airflow apparatus 60. The controller 200 is disposed within the housing 62 and may be mounted on a printed circuit board (PCB) supported by internal structural features of the housing 62. The controller 200 is operably coupled to the flow sensing module 100 and the feedback assembly 300.

The feedback assembly 300 is positioned at an upper portion of the housing 62 and includes both visual and auditory output components configured to provide real-time guidance to a user. The feedback assembly 300 includes a speaker 320 configured to generate audio output and an LED array 350 configured to provide visual indications. The LED array 350 may be arranged in a generally circular configuration and positioned beneath a transparent cover 112, which protects the LED array 350 while permitting emitted light to be visible to the user. The LED array 350 may be arranged in various geometric configurations, including circular, linear, or segmented arrangements. The opening 64 defined by the upper housing portion 62b is sized and shaped to receive and expose the feedback assembly 300, and may include features configured to support, align, and retain the feedback assembly 300 within the housing 62.

The controller 200 is configured to receive sensor data from the flow sensing module 100 representative of airflow through the flow channel 120 and to process the sensor data to determine one or more ventilation parameters, including flow rate, delivered volume, and ventilation rate. The controller 200 is further configured to generate control signals based on the determined ventilation parameters and to communicate the control signals to the feedback assembly 300 to produce corresponding visual and/or auditory outputs.

In some aspects, the LED array 350 includes multiple sets of LEDs configured to provide distinct types of visual guidance and feedback to the user. A first set of LEDs, referred to as guide LEDs, may be configured to emit light in a periodic pattern corresponding to a target ventilation cadence. The periodic pattern may define a timing sequence including an illumination phase, a dimming phase, and a pause phase, collectively corresponding to a target breathing interval. In aspects, the cadence corresponds to a target ventilation rate appropriate for a patient population, such as an adult resuscitation rate.

A second set of LEDs, referred to as user feedback LEDs, may be configured to provide real-time visual feedback corresponding to a measured airflow parameter, such as delivered volume or compression depth of the self-inflating bag 50. The user feedback LEDs may be arranged circumferentially and configured to illuminate progressively in response to increasing airflow or delivered volume, such that a number of illuminated LEDs corresponds to a magnitude of the measured parameter. In aspects, individual LEDs or groups of LEDs correspond to incremental volume ranges, thereby providing a visual representation of delivered volume.

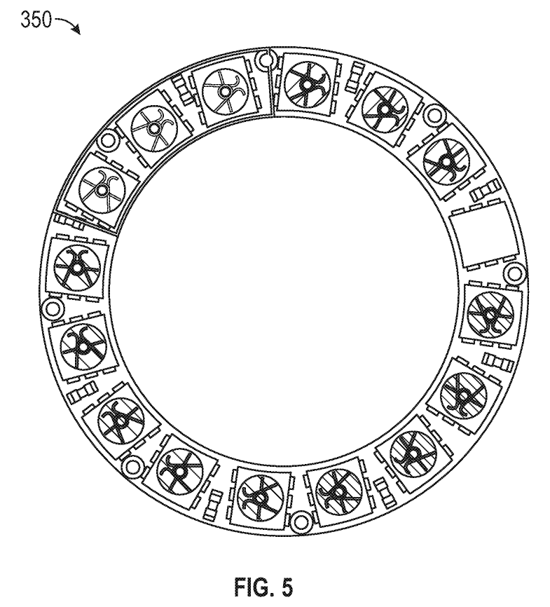

In one embodiment, the user feedback LEDs may include a sequence of indicators having distinct visual characteristics corresponding to predefined thresholds, including a first region indicating insufficient volume delivery, an intermediate region indicating proximity to a target volume, a target region indicating achievement of the desired volume, and an over-delivery region indicating excessive volume delivery. These regions may be distinguished by color, intensity, or illumination pattern, such as white, yellow, green, and red indicators, respectively. In this manner, the LED array 350 provides intuitive, real-time visual guidance to assist the user in delivering ventilation within desired parameters. An example configuration and arrangement of the LED array 350 is illustrated in FIG. 5.

The speaker 320 of the feedback assembly 300 is configured to provide audio output corresponding to operation of the inline airflow apparatus 60. In aspects, the speaker 320 is configured to output one or more instructional audio sequences to guide a user in proper device setup and operation, including instructions for establishing a proper seal and performing ventilation. The speaker 320 is further configured to output corrective feedback signals in response to detected deviations from one or more target ventilation parameters. The corrective feedback signals may include prompts to adjust compression force, ventilation rate, or timing between compressions. In aspects, the corrective feedback is selected based on a type of deviation detected, such as under-delivery, over-delivery, excessive rate, insufficient rate, or incomplete re-expansion of the self-inflating bag 50.

In certain aspects, the controller 200 is configured to detect repeated deviations of a similar type and to cause the speaker 320 to output an escalated or more detailed corrective instruction. Additionally, the speaker 320 may generate confirmation signals, such as tones or audible indicators, in response to achieving a target ventilation parameter.

In aspects, the inline airflow apparatus 60 may further include one or more visual alignment indicators configured to assist the user in establishing a proper seal between the patient interface connector 68 and a patient’s face. For example, a first visual indicator may be disposed on the inline airflow apparatus 60 and a corresponding second visual indicator may be disposed on a mask component, such that proper alignment of the indicators corresponds to correct placement and sealing of the mask. These visual indicators may be implemented as printed images, markings, or other visual cues configured to guide user positioning and reduce air leakage during ventilation.

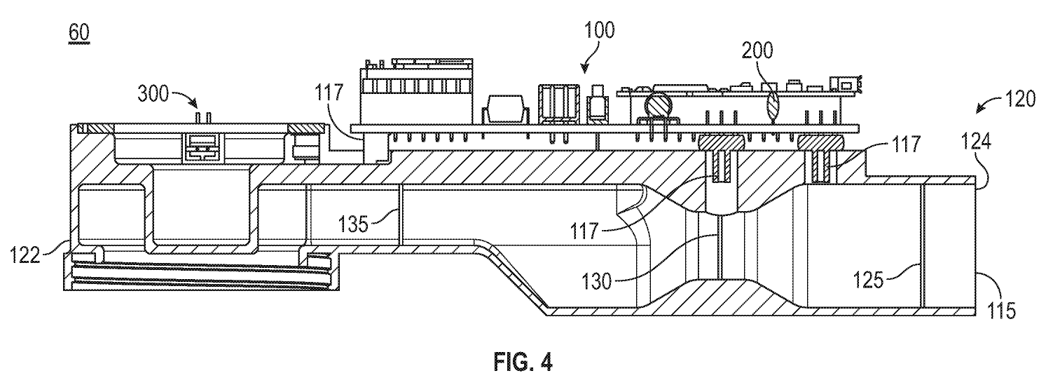

FIG. 4 illustrates a cross-sectional view of the inline airflow apparatus 60, highlighting the internal configuration of the flow channel 120 and the flow sensing module 100 in accordance with aspects of the present disclosure. The flow channel 120 extends between a distal end 124 and a proximal end 122 and is configured to direct airflow from a ventilation source toward a patient interface while enabling measurement of one or more airflow parameters. The flow channel 120 includes an inlet section 125, a converging section 130, and a diverging section 135, which collectively define a geometry configured to generate a pressure differential corresponding to airflow through the inline airflow apparatus 60. In aspects, the geometry of the flow channel 120 is a Venturi geometry.

The inlet section 125 is positioned adjacent the distal end 124 of the flow channel 120 and is configured to receive airflow from a ventilation device via the connection port 115. In aspects, the inlet section 125 is generally cylindrical and has a diameter substantially corresponding to an outlet diameter of the ventilation device, thereby facilitating a smooth transition of airflow into the flow channel 120 with minimal turbulence, separation, or pressure loss. Air entering the inlet section 125 may initially exhibit a relatively lower velocity and higher static pressure relative to downstream regions of the flow channel 120.

The converging section 130 extends from the inlet section 125 toward the proximal end 122 and is configured to reduce a cross-sectional area of the flow channel 120. In aspects, the converging section 130 is of a smaller cross-sectional area than the cross-sectional area of inlet 125. As airflow passes through the converging section 130, the velocity of the airflow increases while the static pressure correspondingly decreases in accordance with Bernoulli’s principle. In aspects, the converging section 130 includes a tapered or conical geometry configured to provide a gradual reduction in diameter, thereby minimizing flow separation and maintaining a predictable pressure gradient suitable for accurate measurement.

The diverging section 135 extends from the converging section 130 toward the proximal end 122 and is configured to gradually increase the cross-sectional area of the flow channel 120. As airflow passes from the converging section 130 into the diverging section 135, the airflow transitions from a region of increased velocity and reduced static pressure to a region of decreasing velocity and recovering pressure. In aspects, the converging section 130 and diverging section 135 collectively define a minimum cross-sectional region therebetween at which airflow velocity is relatively high and static pressure is relatively low.

The flow sensing module 100 is operably coupled to the flow channel 120 and includes a plurality of sensors 117 positioned at selected locations along the flow channel 120. In the illustrated embodiment, a first sensor 117 is positioned to communicate with the inlet section 125, a second sensor 117 is positioned to communicate with the converging section 130, and a third sensor 117 is positioned to communicate with the diverging section 135. Each sensor 117 is configured to measure a parameter indicative of local airflow conditions, such as static pressure, at its respective location. Each sensor 117 is fluidly coupled to the flow channel 120 via a corresponding port, aperture, or conduit extending between the flow channel 120 and the sensor 117, thereby enabling measurement of local static pressure without substantially disrupting airflow. The measured pressures correspond to different airflow velocities at each section of the flow channel 120, wherein an increase in velocity is associated with a decrease in static pressure in accordance with Bernoulli’s principle. The sensors 117 are configured to generate signals corresponding to measured pressures at the inlet section 125, converging section 130, and diverging section 135, and the flow sensing module 100 and/or controller 200 is configured to determine one or more pressure differentials between the sensors 117, the pressure differentials being indicative of airflow through the flow channel 120.

In aspects, the flow channel 120, including the inlet section 125, converging section 130, and diverging section 135, may be formed from stainless steel or other corrosion-resistant materials suitable for repeated use and exposure to humidified or oxygen-enriched airflow. The geometry of the converging section 130 and the diverging section 135 may be selected to minimize energy loss, reduce turbulence, and improve measurement accuracy. For example, in one embodiment, the converging section 130 may have an angle of approximately 21 degrees, while the diverging section 135 may have an angle of approximately 7 degrees, thereby providing controlled acceleration and deceleration of airflow. In some aspects, the flow channel 120 and/or housing 62 may be formed using additive manufacturing techniques, such as resin-based three-dimensional (3D) printing, to enable precise control of internal geometries and rapid prototyping or customization.

In aspects, the dimensions of the flow channel 120 may be selected to be compatible with standard ventilation equipment. For example, the inlet section 125 may have a diameter of approximately 22 mm corresponding to a standard tubing diameter, while the converging section 130 may reduce the diameter to a smaller dimension, such as approximately 12 mm, to generate a measurable pressure differential while maintaining safe airflow characteristics. These dimensions may be selected to balance pressure drop generation, measurement sensitivity, and patient safety.

In certain aspects, the inline airflow apparatus 60 may be adapted for use across different patient populations, including infants, children, adolescents, and adults. Such adaptation may be achieved by modifying one or more geometric characteristics of the flow channel 120, including diameters of the inlet section 125 and converging section 130, angles of the converging section 130 and diverging section 135, and relative lengths of each section, to accommodate different expected airflow ranges and pressure profiles. Additionally, the flow sensing module 100 and associated sensors 117 may be selected or calibrated to operate within pressure ranges appropriate for the intended patient population. In aspects, the controller 200 is configured to store and selectively apply different calibration parameters, target ventilation ranges, and threshold values corresponding to different patient types. For example, the controller 200 may be programmed with distinct parameter sets for adult, pediatric, and infant use, and may select or adjust such parameters based on a user input, device configuration, or predefined operating mode. In this manner, the inline airflow apparatus 60 may be tailored to provide accurate measurement and appropriate feedback across a range of clinical applications.

FIG. 5 illustrates an example configuration of the LED array 350 of the feedback assembly 300 in accordance with aspects of the present disclosure. The LED array 350 is shown as a generally circular arrangement of a plurality of light-emitting elements configured to provide visual guidance corresponding to ventilation parameters. In the illustrated embodiment, the LED array 350 includes one or more guide LEDs configured to indicate a target ventilation cadence and one or more user feedback LEDs configured to represent delivered volume. The guide LEDs may be configured to illuminate in a periodic pattern corresponding to a desired ventilation rate, such that illumination, dimming, and pause intervals collectively define a timing sequence for user actuation of a ventilation device, such as the self-inflating bag 50.

In aspects, the user feedback LEDs are arranged circumferentially and are configured to illuminate progressively in response to increasing delivered volume, thereby providing a visual indication of the magnitude of airflow delivered during a ventilation event. Each LED or group of LEDs may correspond to an incremental volume range, such that illumination of additional LEDs reflects increasing compression of a ventilation device, such as the self-inflating bag 50. A target volume region may be indicated by one or more visually distinct LEDs, and one or more LEDs positioned beyond the target region may be configured to indicate excessive volume delivery. In this manner, the LED array 350 provides an intuitive visual representation of both ventilation cadence and delivered volume.

The specific number, arrangement, color, and illumination behavior of the LEDs shown in FIG. 5 are provided for illustrative purposes only. The LED array 350 may be arranged in a circular, arcuate, linear, segmented, or other geometric configuration, and alternative configurations may be implemented without departing from the scope of the present disclosure. For example, the LED array 350 may be implemented in non-circular arrangements, may utilize different color schemes or intensity variations, and may be configured to represent additional or alternative ventilation parameters.

FIGS. 6–7 illustrate an embodiment 70 in which the inline airflow apparatus 60 is operably coupled to a self-inflating bag 50 in accordance with aspects of the present disclosure. In this configuration, the inline airflow apparatus 60 is positioned between the self-inflating bag 50 and a patient interface connector 68, such that airflow generated by the self-inflating bag 50 passes through the inline airflow apparatus 60 prior to delivery to a patient. The illustrated embodiment 70 represents an exemplary use configuration of the inline airflow apparatus 60 and is not limited to any particular bag or ventilation device.

The self-inflating bag 50 includes a first end 52 and a second end 54. The first end 52 is configured to be removably and sealably coupled to the bag interface port 115 of the inline airflow apparatus 60, thereby establishing a fluid communication pathway between the self-inflating bag 50 and the flow channel 120 of the inline airflow apparatus 60. The second end 54 of the self-inflating bag 50 is coupled to a reservoir interface 55 configured to facilitate intake of air and/or supplemental oxygen. In the illustrated embodiment, the reservoir interface 55 includes an air inlet valve 56 and an oxygen inlet port, which are configured to permit entrainment of ambient air and/or oxygen into the self-inflating bag 50 during expansion. The self-inflating bag 50 is configured to be manually compressed by a user to generate a pressurized airflow. Upon compression, air contained within the self-inflating bag 50 is forced through the first end 52 and into the inline airflow apparatus 60 via the bag interface port 115. Upon release, the self-inflating bag 50 returns toward an expanded state and draws air into the bag through the reservoir interface 55. The self-inflating bag 50 may be formed from an elastomeric or polymeric material configured to provide resilient expansion and repeatable volumetric output during successive compression cycles.

The inline airflow apparatus 60 includes a housing 62 defining an internal flow channel 120 extending between the bag interface port 115 and the patient interface connector 68. The bag interface port 115 is configured to receive the first end 52 of the self-inflating bag 50 in a sealed manner, while the patient interface connector 68 is configured to couple to a face mask, airway device, or other patient interface (not shown). In this manner, the inline airflow apparatus 60 is positioned inline with the airflow path between the self-inflating bag 50 and the patient.

The inline airflow apparatus 60 further includes a flow sensing module 100, a controller 200, and a feedback assembly 300 disposed within or supported by the housing 62. The flow sensing module 100 includes a plurality of sensors 117 configured to measure airflow-related parameters within the flow channel 120, as described with respect to FIG. 4. The controller 200 is operably coupled to the sensors 117 and is configured to receive signals corresponding to airflow conditions within the flow channel 120. The controller 200 processes these signals to determine one or more ventilation parameters, including, for example, flow rate, delivered volume, and ventilation rate. The feedback assembly 300 is operably coupled to the controller 200 and is configured to output visual and/or auditory feedback to a user based on the determined ventilation parameters.

As illustrated in the sectional view of FIG. 7, airflow generated by compression of the self-inflating bag 50 enters the inline airflow apparatus 60 through the bag interface port 115 and flows through the flow channel 120 toward the patient interface connector 68. As airflow passes through the inlet section 125, the converging section 130, and the diverging section 135, a pressure differential is generated across the flow channel 120 and detected by the flow sensing module 100. The controller 200 processes the sensed data to determine ventilation parameters and generates corresponding feedback signals. The feedback assembly 300 communicates this information to the user in real time, thereby assisting the user in delivering ventilation with appropriate cadence and volume. During expansion of the self-inflating bag 50, ambient air and/or supplemental oxygen is drawn into the bag 50 through the air inlet valve 56 and oxygen inlet port 57, respectively, thereby replenishing the bag 50 for subsequent ventilation cycles.

In operation, a user compresses the self-inflating bag 50 to generate airflow, which is directed through the inline airflow apparatus 60. As airflow enters the flow channel 120 through the bag interface port 115, it passes through regions of varying cross-sectional geometry, including the inlet section 125, the converging section 130, and the diverging section 135. These geometric variations cause corresponding changes in airflow velocity and static pressure within the flow channel 120 in accordance with Venturi and Bernoulli principles.

The sensors 117 of the flow sensing module 100 are positioned to measure pressure at selected regions of the flow channel 120, including at least two of the inlet section 125, converging section 130, and diverging section 135. The sensors 117 generate signals representative of local pressure conditions, and the controller 200 is configured to receive these signals and determine one or more pressure differentials between the selected regions. The measured pressure differentials correspond to airflow characteristics within the flow channel 120 and provide a basis for determining ventilation performance. The controller 200 processes the received signals to determine one or more airflow parameters associated with ventilation delivered by the self-inflating bag 50, including flow rate, tidal volume, and ventilation rate. In some aspects, the controller 200 may utilize one or more calibration relationships, lookup tables, or empirically derived coefficients to correlate measured pressure differentials to volumetric airflow and delivered tidal volume. In aspects, the controller 200 executes algorithms configured to convert pressure measurements into volumetric flow estimates, integrate flow over time to determine delivered volume, and analyze temporal variations in the sensor signals to determine ventilation cadence. The controller 200 may further analyze pressure values to detect abnormal conditions, such as insufficient sealing or airway obstruction

In aspects, controller 200 applies one or more calibration coefficients, compensation algorithms, and/or filtering techniques to improve measurement accuracy. Such techniques may account for variations in flow conditions, user compression technique, environmental conditions, and manufacturing tolerances of the flow channel 120. For example, the controller 200 may apply digital filtering, such as moving average or low-pass filtering, to reduce signal noise, and may utilize stored calibration data to adjust calculated airflow parameters. These processing techniques enable consistent and reliable determination of ventilation parameters across a range of operating conditions.

The calculated airflow parameters are communicated by the controller 200 to the feedback assembly 300, which is configured to provide real-time feedback to the user. The feedback assembly 300 may include a speaker 320 configured to generate audible feedback and an LED array 350 configured to provide visual feedback indicative of ventilation performance. In aspects, the LED array 350 is arranged in a ring configuration and is configured to visually represent parameters such as delivered volume and ventilation cadence, while the speaker 320 provides instructional or corrective audio prompts. In this manner, the flow channel 120, in combination with the sensors 117 and the controller 200, provides a compact, inline, and non-invasive mechanism for monitoring ventilation performance. The inline airflow apparatus 60 enables measurement of airflow parameters directly within the ventilation pathway without requiring external flow meters or additional instrumentation, thereby facilitating integration with standard self-inflating bag systems while maintaining a low-profile form factor and minimizing disruption to airflow.

Upon release of the self-inflating bag 50, the bag expands and draws air into the interior volume through the air inlet valve 56 and/or oxygen inlet port of the reservoir interface 55. This replenishes the self-inflating bag 50 for a subsequent ventilation cycle. The inline airflow apparatus 60 continuously measures airflow conditions during successive compression and expansion cycles and provides real-time feedback to guide the user in maintaining desired ventilation parameters.

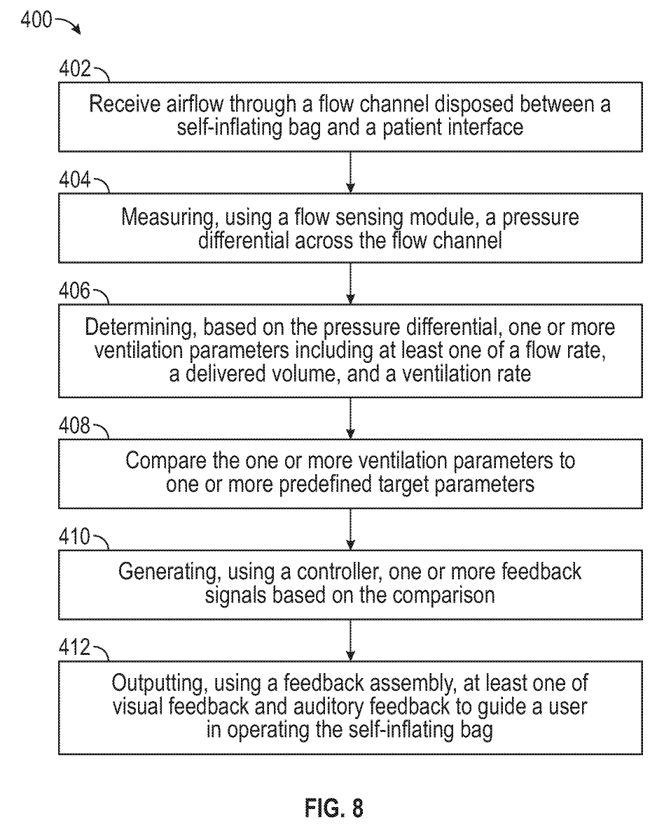

Referring now to FIG. 8, a method 400 for assisting ventilation using an inline airflow apparatus 60 (FIGS. 3-6) is illustrated in accordance with aspects of the present disclosure. In various aspects, method 400 is executed by controller 200 in communication with flow sensing module 100 and feedback assembly 300 to monitor airflow through inline airflow apparatus 60 and provide real-time feedback to a user operating self-inflating bag 50. Method 400 may be performed by a local embedded controller disposed within housing 62, such as a microcontroller mounted on a printed circuit board within inline airflow apparatus 60, or in alternative aspects by another computing device in wired or wireless communication with the system. The method 400 may be implemented during emergency ventilation, training, simulation, or other manual respiratory support scenarios.

Prior to initiation of active ventilation monitoring, a user positions the inline airflow apparatus 60 for use. In aspects, inline airflow apparatus 60 is coupled to self-inflating bag 50 through bag interface port 115, and patient interface connector 68 is coupled to a face mask or airway interface configured to be placed on a patient. The user may activate the inline airflow apparatus 60 by actuating power switch 65, thereby electrically coupling power source 110 to controller 200, flow sensing module 100, and feedback assembly 300.

In aspects, activation of the inline airflow apparatus 60 causes feedback assembly 300 to output an instructional guidance sequence. For example, speaker 320 may provide one or more audio prompts informing the user how to operate the inline airflow apparatus 60 and perform ventilation. In some aspects, the instructional guidance includes instructions related to establishing a proper seal between the patient interface and the patient’s face. As described previously, the inline airflow apparatus 60 may include one or more visual alignment indicators, such as a first visual indicator disposed on inline airflow apparatus 60 and a second corresponding visual indicator disposed on a mask component. The user may align the visual indicators to facilitate proper positioning and sealing of the mask and reduce air leakage during ventilation.

At block 402, airflow is received through flow channel 120 disposed between self-inflating bag 50 and a patient interface. In operation, the user compresses self-inflating bag 50 with a hand or hands to expel air from the bag through first end 52 and into bag interface port 115. The airflow enters flow channel 120 and travels toward patient interface connector 68 for delivery to the patient. During this process, the airflow passes through inlet section 125, converging section 130, and diverging section 135, which together define a geometry configured to produce predictable changes in pressure as airflow moves through inline airflow apparatus 60. In aspects employing a Venturi geometry, the airflow is accelerated in converging section 130 and pressure is correspondingly reduced relative to inlet section 125. The airflow then passes through diverging section 135, where the pressure partially recovers before exiting toward the patient interface. During release of self-inflating bag 50, the bag re-expands and air is drawn into the bag via air inlet valve 56 and/or oxygen inlet port 57 for subsequent ventilation cycles.

At block 404, a pressure differential across flow channel 120 is measured using flow sensing module 100. In aspects, flow sensing module 100 includes one or more sensors 117 configured to measure pressure at selected positions along flow channel 120, such as within inlet section 125 and converging section 130. The sensors 117 may comprise analog or digital pressure sensors, including MEMS sensors, differential pressure sensors, piezoresistive transducers, capacitive sensors, or other pressure-responsive devices. As airflow propagates through converging section 130, a pressure drop is generated relative to inlet section 125. The pressure signals corresponding to these locations are sensed and communicated to controller 200. In aspects, controller 200 performs signal conditioning operations including filtering, amplification (e.g., via one or more amplification stages such as instrumentation amplifiers), and analog-to-digital conversion to generate usable digital representations of the sensed pressure values. The controller 200 may sample the pressure data at a rate sufficient to characterize transient airflow behavior produced during manual compression and release of self-inflating bag 50.

At block 406, controller 200 determines one or more ventilation parameters based on the measured pressure differential. In aspects, controller 200 computes an airflow rate using a relationship derived from Bernoulli’s principle and continuity relationships, taking into account the geometry of flow channel 120, including inlet section 125 and converging section 130. Controller 200 may further integrate the determined airflow rate over time to determine a delivered volume associated with a given ventilation event. The controller 200 may also determine a ventilation rate based on a time interval between successive ventilation events. In this manner, the measured differential pressure is translated into ventilation parameters including, for example, flow rate, delivered volume, and breaths per minute. n aspects, the computation may further incorporate stored calibration coefficients or empirically derived relationships to improve accuracy of volumetric estimation across varying operating conditions.

In aspects, controller 200 is further configured to identify individual squeeze events and release events based on time-varying sensor signals. For example, controller 200 may process a signal representative of airflow or bag actuation and determine transitions between compression and release phases. In certain aspects, controller 200 may utilize signal processing techniques, such as comparison of a current signal value to a running average or threshold, to determine whether the user is actively compressing the self-inflating bag 50 or allowing the bag to re-expand. The controller 200 may detect transitions between these states to identify a time point at which a squeeze is completed and a magnitude associated with the squeeze. In aspects, such operations may be performed using airflow-derived signals from flow sensing module 100. In alternative aspects, such operations may be performed using analog input data from a compression-responsive sensor, such as a potentiometer. The time difference between successive detected squeeze events may be used to determine ventilation rate, while the magnitude of each event may be used to determine delivered volume and/or compression depth. In some embodiments, determining the one or more ventilation parameters further comprises selecting or applying one or more calibration parameters, target ventilation ranges, or threshold values corresponding to a selected patient population, such as adult, pediatric, or infant use.

At block 408, controller 200 compares the determined ventilation parameters to one or more predefined target parameters stored in memory. Controller 200 may be configured to execute program instructions stored in memory to perform such comparisons. In aspects, the target parameters include a target ventilation cadence and a target delivered volume selected for a patient population or clinical use case. For example, in one embodiment, a target ventilation cadence may be approximately 12 breaths per minute for adult resuscitation. In aspects, the target cadence is visually represented as an illumination cycle in which one or more LEDs increase in illumination for approximately one second, decrease in illumination for approximately one second, and remain off for approximately three seconds, collectively corresponding to a desired ventilation interval. In aspects, a target delivered volume may be approximately 500 mL for an adult patient. In other aspects, the target parameters may be adjusted based on patient size, age, or clinical condition. Controller 200 may compare determined values against one or more thresholds to identify conditions including under-delivery, over-delivery, excessive ventilation rate, insufficient ventilation rate, or incomplete refill of self-inflating bag 50.

In aspects, controller 200 classifies measured performance into one or more categories based on the comparison performed at block 408. For example, a delivered volume below a lower threshold may be classified as insufficient volume delivery, while a delivered volume above an upper threshold may be classified as excessive volume delivery. A ventilation rate above a target range may be classified as excessively fast, and a ventilation rate below a target range may be classified as too slow. The controller 200 may further determine whether the self-inflating bag 50 has been allowed to sufficiently re-expand between squeezes. These classifications may be performed using threshold comparisons, conditional logic, lookup tables, or other decision routines executed by controller 200. In aspects, controller 200 tracks repeated occurrences of a same classification across multiple successive ventilation cycles.

In aspects, comparing the one or more ventilation parameters to predefined target parameters further comprises identifying abnormal airflow conditions based on measured pressure values or pressure differentials. For example, a measured pressure or pressure differential below an expected threshold may indicate an insufficient seal between the patient interface and the patient, while a measured pressure or pressure differential above an expected threshold may indicate an airway obstruction or blockage. The controller 200 may be configured to classify such conditions and to generate corresponding feedback signals to alert the user and prompt corrective action.

At block 410, controller 200 generates one or more feedback signals based on the comparison and classification performed in block 408. The feedback signals may include visual guidance signals, auditory instruction signals, corrective feedback signals, confirmation signals, or escalated feedback signals. In aspects, controller 200 generates visual cadence signals corresponding to a target ventilation interval and visual volume signals corresponding to measured volume output. Controller 200 may further generate auditory prompts instructing a user to modify squeeze force, ventilation rate, or refill timing. In certain aspects, if a same deviation is detected across multiple successive ventilation cycles, controller 200 generates an escalated feedback signal corresponding to a more descriptive corrective instruction.

At block 412, feedback assembly 300 outputs at least one of visual feedback and auditory feedback based on the generated feedback signals. In aspects, LED array 350 provides visual instruction and visual feedback using multiple sets of LEDs. A first set of LEDs, referred to as guide LEDs, may be configured to illuminate according to a target ventilation cadence. In one embodiment, the guide LEDs are white LEDs timed to illuminate at a rate corresponding to a desired breathing interval, such as approximately once every four seconds. In aspects, the guide LEDs may increase in illumination, decrease in illumination, and pause to represent a complete ventilation cycle. This visual cadence instruction may be coordinated with a simultaneous audio prompt emitted by speaker 320 instructing the user to squeeze in accordance with the indicated timing.

A second set of LEDs, referred to as user feedback LEDs, may be configured to represent measured delivered volume in real time. In aspects, the user feedback LEDs illuminate progressively as volume output increases during a squeeze event, thereby providing a dynamic visual representation of delivered volume. In one embodiment, individual LEDs or groups of LEDs correspond to incremental delivered volume amounts. In aspects, the user feedback LEDs include a sequence of color-coded indicators, such as one or more white LEDs indicating initial compression, a yellow LED indicating proximity to a target volume, a green LED indicating achievement of a target volume, and a red LED indicating excessive compression. In aspects, the target delivered volume is visually indicated by a designated LED position or color marker. The mapping between LEDs and delivered volume may be adjusted for different patient populations.

Auditory feedback is provided to the user by speaker 320. In aspects, speaker 320 provides multiple forms of audio output. A first form of audio output may include instructional prompts that guide the user in operating the inline airflow apparatus 60 and performing ventilation. A second form of audio output may include corrective feedback prompts generated during operation in response to detected deviations from target parameters. For example, if the measured delivered volume is below a threshold, the speaker 320 may output a prompt such as “squeeze harder,” and if the measured delivered volume is above a threshold, the speaker 320 may output a prompt such as “squeeze softer.” If the measured ventilation rate is above a target range, the speaker 320 may output a prompt such as “squeeze slower,” and if the measured ventilation rate is below a target range, the speaker 320 may output a prompt such as “squeeze faster.” In aspects, additional prompts may instruct the user to allow the self-inflating bag 50 to fully re-expand prior to a subsequent compression.

In aspects, if a same deviation is detected repeatedly across multiple successive ventilation cycles, controller 200 causes speaker 320 to output a more detailed corrective instruction. A further form of audio output may comprise a confirmation signal, such as a tone, emitted when a desired ventilation parameter is achieved. In this manner, feedback assembly 300 provides real-time guidance and feedback to assist a user in delivering ventilation in accordance with target parameters.

In aspects, if any of the above mistakes are detected repeatedly, such as three times in succession, controller 200 causes speaker 320 to output a longer, more descriptive corrective instruction. Such escalated feedback may provide additional explanatory guidance regarding how to correct the detected error, such as adjusting hand pressure, slowing cadence, waiting for bag refill, or re-establishing mask seal. A third form of audio output may comprise a confirmation signal, such as a tone or ding, emitted when the user has reached a desired volume threshold or otherwise performed within target ventilation parameters. In this manner, feedback assembly 300 provides both anticipatory guidance and responsive feedback to coach the user toward proper ventilation performance.

Method 400 may be repeated continuously over successive ventilation cycles while the user manually operates self-inflating bag 50. For each cycle, controller 200 senses airflow-related data, determines ventilation parameters, compares the parameters to stored target values, generates corresponding feedback signals, and outputs visual and/or auditory guidance through feedback assembly 300. This process may continue until manual ventilation is no longer required, the inline airflow apparatus 60 is powered off using power switch 65, or a termination condition is otherwise satisfied. Because method 400 provides real-time coaching using measured airflow characteristics and structured feedback outputs, the inline airflow apparatus 60 enables users of varying skill levels to deliver ventilation more consistently and within desired safety and efficacy ranges.

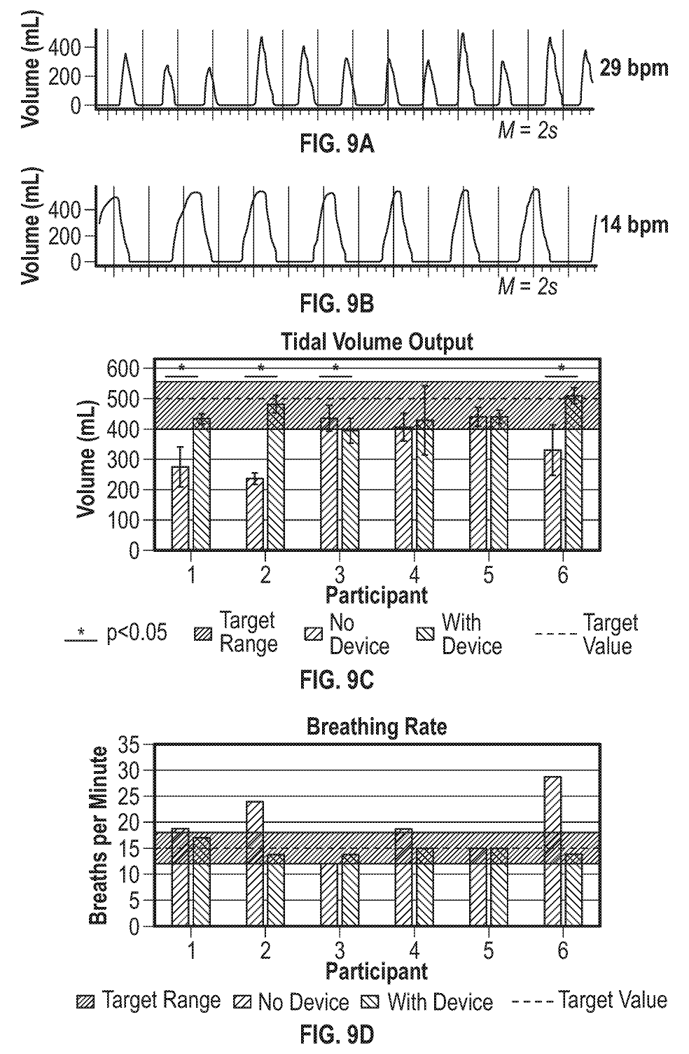

FIGS. 9A–9D illustrate experimental results demonstrating performance characteristics of the inline airflow apparatus 60 during use with a self-inflating bag 50. The results were obtained using a test lung model configured to simulate respiratory conditions and to measure ventilation parameters, including tidal volume and breathing rate, during manual operation of the self-inflating bag 50 by one or more users. FIG. 9A illustrates a representative waveform of delivered volume over time during manual ventilation without use of the inline airflow apparatus 60. As shown, the waveform exhibits irregular timing and inconsistent peak amplitudes, corresponding to variability in both breathing rate and delivered tidal volume. In the illustrated example, the measured breathing rate is approximately 29 breaths per minute (bpm), which exceeds a target ventilation range for adult resuscitation. FIG. 9B illustrates a representative waveform of delivered volume over time during manual ventilation with the inline airflow apparatus 60 coupled to the self-inflating bag 50. In contrast to FIG. 9A, the waveform demonstrates more consistent timing and more uniform peak amplitudes, indicating improved control of both breathing rate and delivered tidal volume. In the illustrated example, the measured breathing rate is approximately 14 bpm, which falls within a desired target range for adult ventilation.

FIG. 9C illustrates a comparison of average tidal volume output across multiple users during a testing interval, both with and without use of the inline airflow apparatus 60. Each bar represents an individual user, and the shaded region indicates a target tidal volume range. As shown, use of the inline airflow apparatus 60 results in tidal volume outputs that more consistently fall within the target range, demonstrating improved user performance and reduced variability relative to unaided ventilation. FIG. 9D illustrates a comparison of average breathing rate across multiple users during a testing interval, both with and without use of the inline airflow apparatus 60. As shown, users operating without the inline airflow apparatus 60 tend to exceed the target breathing rate range, whereas users operating with the inline airflow apparatus 60 achieve breathing rates that fall within or closer to the target range. In aspects, improvements observed with use of the inline airflow apparatus 60 may be statistically significant, indicating that the feedback provided by the inline airflow apparatus 60 assists users in achieving desired ventilation parameters. These results demonstrate a technical improvement in user-guided ventilation control, wherein the inline airflow apparatus 60 enables real-time modulation of user input through feedback-driven correction, thereby reducing variability in ventilation rate and improving adherence to target respiratory parameters without requiring specialized training or external monitoring equipment.

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

As used herein, the phrases “in an aspect,” “in aspects,” “in some aspects,” “in various aspects,” or similar expressions can refer to one or more of the same or different aspects and do not necessarily refer to mutually exclusive aspects.

The phrases “in an embodiment,” “in embodiments,” “in various embodiments,” “in some embodiments,” or “in other embodiments” may each refer to one or more of the same or different example embodiments provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A method of assisting ventilation using an inline airflow apparatus, comprising:
receiving airflow through a flow channel disposed between a self-inflating bag and a patient interface;
measuring, using a flow sensing module, a pressure differential across the flow channel;
determining, based on the pressure differential, one or more ventilation parameters including at least one of a flow rate, a delivered volume, and a ventilation rate;
comparing the one or more ventilation parameters to one or more predefined target parameters;
generating, using a controller, one or more feedback signals based on the comparison; and
outputting, using a feedback assembly, at least one of visual feedback and auditory feedback to guide a user in operating the self-inflating bag.

2. The method of claim 1, wherein determining the delivered volume comprises calculating volume based on the measured pressure differential across the flow channel.

3. The method of claim 1, wherein the visual feedback comprises illuminating a plurality of LEDs in a progressive manner corresponding to increasing delivered volume.

4. The method of claim 3, wherein each LED corresponds to a predefined volume increment.

5. The method of claim 3, wherein the visual feedback further includes indicating a target volume range using at least one visually distinct LED.

6. The method of claim 1, wherein the visual feedback further comprises generating a periodic illumination pattern corresponding to a target ventilation cadence.

7. The method of claim 6, wherein the periodic illumination pattern includes an illumination phase, a dimming phase, and a pause phase corresponding to a predefined breathing interval.

8. The method of claim 1, wherein the auditory feedback comprises generating one or more instructional prompts.

9. The method of claim 8, wherein the instructional prompts include guidance to adjust at least one of compression force, ventilation rate, or timing between compressions.

10. The method of claim 1, further comprising detecting a deviation from a target ventilation parameter and generating corrective feedback corresponding to the deviation.

11. The method of claim 10, wherein the corrective feedback includes at least one of:
indicating insufficient volume delivery;
indicating excessive volume delivery;
indicating a ventilation rate above a threshold;
indicating a ventilation rate below a threshold;
indicating an insufficient seal based on a measured pressure or pressure differential below an expected threshold; or
indicating an airway obstruction based on a measured pressure or pressure differential above an expected threshold.

12. The method of claim 1, further comprising detecting repeated deviations of a same type and generating an escalated feedback instruction.

13. The method of claim 1, further comprising generating a confirmation signal upon achieving a target ventilation parameter.

14. An inline airflow apparatus for assisting ventilation, comprising:
a housing defining a flow channel configured to be fluidly coupled between a self-inflating bag and a patient interface;
a flow sensing module comprising a plurality of sensors positioned along the flow channel and configured to measure pressure at one or more locations within the flow channel;
a feedback assembly comprising at least one visual output device and at least one auditory output device;
at least one processor; and
at least one memory storing instructions, which when executed by the at least one processor, cause the apparatus to:
receive airflow through the flow channel;
measure, using the flow sensing module, a pressure differential across the flow channel;
determine, based on the pressure differential, one or more ventilation parameters including at least one of a flow rate, a delivered volume, and a ventilation rate;
compare the one or more ventilation parameters to one or more predefined target parameters;
generate one or more feedback signals based on the comparison; and
output, using the feedback assembly, at least one of visual feedback and auditory feedback to guide a user in operating the self-inflating bag.

15. The inline airflow apparatus of claim 12, wherein the flow channel comprises a geometry including an inlet section, a converging section, and a diverging section configured to generate the pressure differential.

16. The inline airflow apparatus of claim 15, wherein the plurality of sensors are positioned to measure pressure at at least two of the inlet section, the converging section, and the diverging section.

17. The inline airflow apparatus of claim 12, wherein the one or more predefined target parameters include a target ventilation rate and a target delivered volume.

18. The inline airflow apparatus of claim 12, wherein the feedback assembly comprises an LED array configured to provide visual feedback indicative of the delivered volume.

19. The inline airflow apparatus of claim 18, wherein the LED array is configured to illuminate progressively in response to increasing delivered volume and to indicate when a target volume has been reached.

20. The inline airflow apparatus of claim 12, wherein the instructions further cause the apparatus to determine a ventilation rate based on temporal variations in the measured pressure differential and to generate feedback signals corresponding to the ventilation rate.
